(19) United States
(12) Patent Application Publication
(10) Pub. No.: US 2025/0221865 A1
(43) Pub. Date: Jul. 10, 2025

(54) Tampon Applicator Made With A Single Piece Barrel and Tampon Product Therefrom

(71) Applicant: Kimberly-Clark Worldwide, Inc., Neenah, WI (US)

(72) Inventors: Ryan Weaver, Appleton, WI (US); Dale K. Ware, Lynchburg, VA (US); Matthew J. Grafton, Clyde, NY (US)

(21) Appl. No.: 18/848,966

(22) PCT Filed: Apr. 29, 2022
(86) PCT No.: PCT/US2022/026963

§ 371 Date: Sep. 20, 2024

(51) Int. Cl. A61F 13/26

(52) U.S. Cl. CPC A61F 13/266

(57) ABSTRACT

A tampon product is disclosed including a tampon applicator designed to expel in a controlled manner a tampon. The tampon applicator is designed in a two-piece construction including a barrel and a plunger. Both the barrel and the plunger can be formed from a single layer of a single polymer material. The tampon applicator, for instance, can include a grip region that not only facilitates injection molding but also has characteristics that have been found well suited for being held and manipulated by a user.

### BACKGROUND

Vaginal tampons are disposable absorbent articles sized and shaped (e.g., cylindrical) for insertion into a women's vaginal canal for absorption of body fluids generally discharged during the woman's menstrual period. Insertion of the tampon into the vaginal canal is commonly achieved using a tampon applicator that comes initially assembled with the tampon. The applicator, which is often made of plastic or cardboard, is disposable. Thus, after the applicator has been used to insert the tampon into the user's vaginal canal the applicator is discarded.

Tampon applicators are typically of a two-piece construction, including a barrel in which the tampon is initially housed and a plunger moveable telescopically relative to the barrel to push the tampon out of the barrel and into the vaginal canal. The barrel has a tip that generally retains the tampon within the barrel until pushed through the tip by the plunger. In normal use, the applicator and more particularly the barrel of the applicator is held by the user by gripping one portion of the barrel (e.g., toward the trailing or plunger end of the barrel) and inserting the barrel, tip end first, into the vaginal canal. The barrel is pushed partially into the canal so that a portion (e.g., toward the leading or exit end of the tampon barrel) is disposed within the vaginal canal and is contact with the walls lining the canal. The plunger is then used to push the tampon out through the tip of the barrel and into the canal. The plunger and barrel are then removed from the vaginal canal, leaving the tampon in place.

In such use, the barrel (and plunger) of the applicator comes into contact with different body parts and/or skin regions of the user, such as the tip and part of the barrel contacting the walls lining the vaginal canal, while the user's finger(s) contact the barrel (and plunger) to grip and hold the barrel and to operate the plunger. The ability of the user to have a secure grip on the applicator while allowing the applicator to comfortably slide into and out of the vaginal canal is thus an important factor in acceptance of the applicator.

In view of the above, in the past, a portion of the barrel has been overmolded with an elastomeric material in order to improve the grip properties of the barrel. Although applying an elastomeric material to the barrel has provided great improvements in the handling and manipulation of the applicator, the elastomeric material not only adds significant cost to the overall product, but is also typically made from materials that are not biodegradable or capable of entering the recycling stream. Thus, applicators overmolded with an elastomeric material are generally not flushable.

Recently, significant importance has been placed on producing tampon applicators from recyclable, biodegradable, and/or flushable materials. Using recyclable, biodegradable, and/or flushable materials to produce the tampon applicator, not only produces an overall sustainable product, but also, in some applications, allows the user to dispose of the tampon applicator in a toilet bowl, which provides the user with cleanliness and discretion.

In view of the above, a need currently exists for a tampon applicator design that is not only easy to grip and manipulate but is also constructed without the use of an elastomeric material. A need also exists for a tampon applicator design that can be made from a single polymer material or composition and formed in a single molding step. A tampon applicator design that meets the above criteria would not only be easy to hold and manipulate but may also be made exclusively from recyclable, biodegradable, and/or flushable materials.

### SUMMARY

In general, the present disclosure is directed to a tampon applicator and to a tampon product incorporating the applicator. In accordance with the present disclosure, the applicator can be made from a single material and can be made with a shape that is easy to hold and grasp without the necessity of overmolding the applicator with a second material, such as an elastomeric material. In this regard, the tampon applicator of the present disclosure is well suited to being formed from a single layer of all types of materials including paperboard materials and polymer materials, including a single layer of an elastomeric polymer. In one aspect, the tampon applicator can be formed from bio-based materials, biodegradable materials, flushable materials, and/or recyclable materials.

In one embodiment, for instance, the present disclosure is directed to a tampon applicator having an elongated barrel that defines an interior chamber for housing a tampon. The barrel can include an inner end spaced longitudinally from an outer end. The tampon applicator can further include a plunger that extends into the outer end of the barrel and is movable relative to the barrel to expel a tampon through the inner end. The barrel can further include a grip region that is positioned at one end of the barrel. In accordance with the present disclosure, the grip region has a configuration that is easy for a user to hold and manipulate. More particularly, the grip region can include a first end and a second and opposite end that coincides with the inner end of the barrel. The second end defines a grip ring. The grip region gradually tapers from the first end to the grip ring. For example, the barrel can define a longitudinal axis and the grip region can gradually taper at an angle to the longitudinal axis of from about 0.5° to about 5°, such as from about 1° to about 3°, such as from about 1.5° to about 2.5°.

In accordance with the present disclosure, the elongated barrel including the grip region can be made from a single piece of material and/or a single layer of material. In one embodiment, for instance, the entire tampon applicator is made from the same polymer composition.

As described above, the grip region includes a grip ring. The grip ring can flare outwardly from the grip region at an angle of from about 7° to about 17°, such as from about 10° to about 14°, such as from about 11° to about 13° in relation to the longitudinal axis of the barrel. In one embodiment, the grip region can further include a raised pattern that has been molded into the surface of the grip region. The raised pattern can facilitate gripping of the grip region. The raised pattern, for instance, can be formed from geometrical elements or can be decorative.

In one aspect, the grip region can include a plurality of internal ribs that define a guide channel having an effective diameter. The plurality of internal ribs can have a leading end and a trailing end. The internal ribs, for instance, can have a height greater at the leading end than at the trailing end. In this regard, the internal ribs can taper in height from the leading end to the trailing end. The internal ribs can also taper in width from the trailing end to the leading end. In one embodiment, the grip region can include greater than about 4 internal ribs, such as having 5 internal ribs, 6 internal ribs, or 7 internal ribs.

The plunger of the tampon applicator can include a finger-contacting end and a tampon-contacting end. The finger-contacting end can define a finger flare, while the tampon-contacting end can define a tampon flare. The tampon flare can have an effective diameter that is greater than the effective diameter of the guide channel. In this manner, the plunger can remain movably engaged within the barrel.

The barrel of the tampon applicator, in one embodiment, can include at least two petals that each have a base and extend longitudinally from the barrel to a tip of the petal. The petal tips can define the longitudinal inner end of the barrel. The petals can be configured relative to each other and to the barrel to assume a closed configuration in which the petals generally close the inner end of the barrel and an open position in which the petals are moved generally transversely outward to form an exit opening at the inner end of the barrel upon movement of the plunger to expel a tampon contained in the barrel through the inner end. In one aspect, the tampon applicator can include 5 petals or 6 petals located at the inner end.

The tampon applicator can be made from any suitable material, such as a paperboard or thermoplastic polymer. In one embodiment, the tampon applicator can be made from a bio-based polymer. The bio-based polymer, for instance, can comprise polylactic acid, a polyhydroxyalkanoate, or mixtures thereof. In one aspect, the tampon applicator can be made from an elastomeric polymer.

The present disclosure is also directed to a tampon product. The tampon product includes a tampon applicator as described above containing a tampon pledget. The tampon pledget can be connected to a drawstring that extends through the plunger of the tampon applicator. In one embodiment, the pledget can be made from natural fibers, such as cotton fibers.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a perspective view of one embodiment of a tampon product made in accordance with the present disclosure;

FIG. 2 is a cross-sectional view of the tampon product illustrated in FIG. 1;

FIG. 3 is a side view of a grip region for a tampon applicator made in accordance with the present disclosure;

FIG. 4A is a partial side view of another embodiment of a grip region for a tampon applicator made in accordance with the present disclosure;

FIG. 4B is a partial side view of still another embodiment of a grip region for a tampon applicator made in accordance with the present disclosure;

FIG. 5 is a partial side view of another embodiment of a grip region for a tampon applicator made in accordance with the present disclosure;

FIG. 6 is a perspective view of a grip region for a tampon applicator made in accordance with the present disclosure illustrating a plurality of elongated ribs;

FIG. 7 is a cross-sectional view of the grip region illustrated in FIG. 6;

FIG. 8 is a perspective view of one embodiment of a plunger that can be incorporated into the tampon applicator of the present disclosure; and

FIG. 9 is a partial cross-sectional view of the finger-contacting end of the plunger illustrated in FIG. 8.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a tampon applicator and to a tampon product incorporating the applicator. The tampon applicator of the present disclosure can include an elongated barrel for housing a tampon and a plunger that a user can use for inserting the tampon into a vaginal canal. In accordance with the present disclosure, the tampon applicator, and particularly the elongated barrel, have a shape and design that allows for the tampon applicator to be made entirely from the same material, such as a polymer or a paperboard. More particularly, the elongated barrel of the tampon applicator has a uniquely designed gripping region that facilitates grasping, holding and manipulating the tampon applicator when a tampon is being inserted. Of particular advantage, the design of the gripping region facilitates holding of the tampon applicator without having to overmold with a different material, such as an elastomeric material, to the gripping region in order to improve the functional properties of the applicator.

The shape and design of the tampon applicator provide numerous advantages and benefits. For example, as described above, the tampon applicator has a shape that is easy to hold and manipulate. In addition, the barrel of the tampon applicator can be made in a single molding process and from a single polymer composition and/or single layer of material that can greatly enhance sustainability. For example, by being made from a single polymer material, the tampon applicator can be formed entirely from bio-based polymers or recycled materials, such as a paperboard or polymer. In one aspect, for instance, the entire tampon applicator can be made from a flushable polymer that quickly degrades when flushed down a toilet or otherwise contacted with moisture.

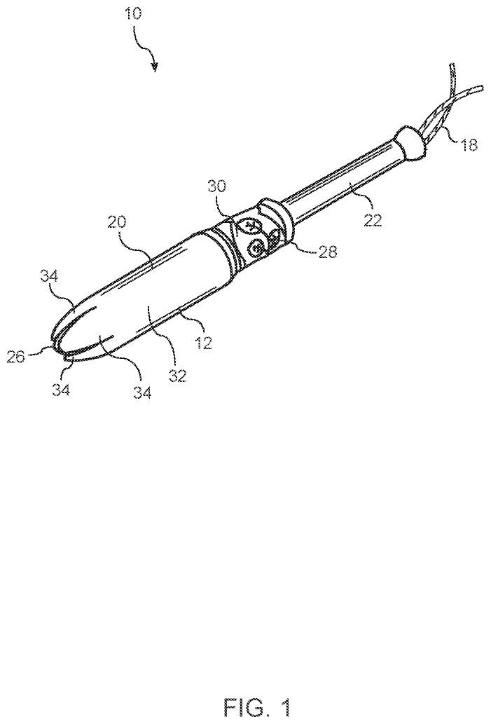

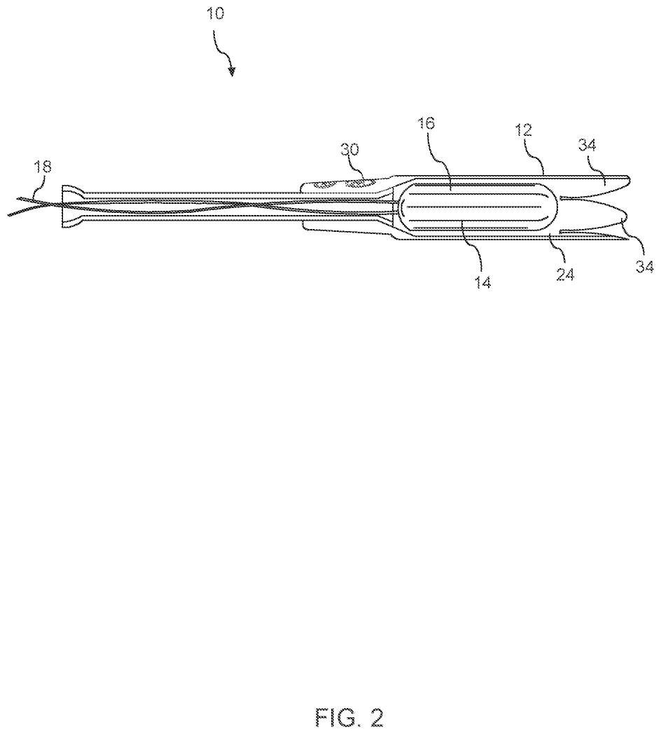

Referring to FIGS. 1-9, embodiments of a tampon product and tampon applicator made in accordance with the present disclosure are shown. Referring to FIGS. 1 and 2, a tampon product 10 is shown that includes a tampon applicator 12 that contains a tampon 14. The tampon 14 includes a tampon pledget 16 attached to a withdrawal string 18. In one embodiment, the tampon pledget 16 can be made from all natural materials. For example, in one embodiment, the tampon pledget 16 can be made from cotton fibers alone or in combination with other natural or synthetic fibers. The withdrawal string 18 is fastened to the tampon pledget 16 generally adjacent to a trailing end thereof. The withdrawal string 18 is for pulling the tampon from the vaginal canal after use for disposal.

The tampon applicator 12 comprises an elongated barrel 20 that houses the tampon pledget 16. The tampon applicator 12 further includes a plunger 22. The plunger 22 is movable telescopically relative to the barrel 12 in order to expel the tampon 14 from the barrel 12. The tampon 14 has a size and shape for insertion into the vaginal canal of a female user to absorb menses, blood and other bodily fluids. It should be understood, however, that the tampon applicator 12 may be used in conjunction with other suitable types of tampons.

In the embodiment illustrated, the tampon applicator 12 includes a longitudinal axis that extends parallel to the length of the barrel 20. The plunger 22 can be placed in a coaxial relationship with the barrel 20 along the longitudinal axis.

The barrel 20 of the tampon applicator 12 is suitably sized and shaped for housing the tampon 14 within an interior chamber 24 of the barrel 12 as shown in FIG. 2. The barrel 20 is also suitably sized and shaped for insertion into a body cavity of the user, such as the vaginal canal of a female user when the tampon is a vaginal tampon. The barrel 20 is generally elongated and also cylindrically shaped having an inner end 26 and an outer end 28. In accordance with the present disclosure, the barrel 20 further includes a uniquely designed grip region 30 adjacent the outer end 28 of the barrel 20. The barrel 20 further includes a central region 32 that is adjacent the grip region 30 and at least in part defines the interior chamber 24. The central region 32 is also adjacent the inner end 26 of the barrel 20. The inner end 26 and the outer end 28 as used herein are referenced relative to the orientation of the tampon applicator 12 and its various components during use thereof, with the barrel 20 being inserted, inner end 26 first, into a body cavity, such as a vaginal canal.

In one embodiment, the inner end 26 of the barrel 20 can include a plurality of extensions or petals 34. The petals 34 are separated by longitudinal slots or spaces. Each of the petals 34 extend longitudinally from a base of the petal 34, where the petal is connected to and more suitably formed integrally with the rest of the barrel 20. As shown in FIG. 1, the petals 34 extend over an opening defined at the inner end 26. The width of each petal 34 can taper inwardly from its base towards its tip. The petals 34 are suitably configured in this manner to permit the petals to be bent inwardly during manufacture of the tampon applicator 12 in order to generally close the barrel 20 at the inner end 26 and enclose the tampon 14 within the interior chamber 24. Consequently, the petals 34 are well suited to, on one hand, assembling the tampon product 12 during manufacture. The slots or spaces between the petals 34, for instance, allow for bending of the petals 34 into the closed configuration as shown in FIG. 1 during manufacture and for flexing or bending transversely and outwardly upon application of force by the tampon 14 when the tampon 14 is guided out of the barrel 20 by the plunger 22.

The petals 34 can have any suitable length and width as measured from the base to the tip in order to provide flexibility in packaging the tampon and in releasing the tampon from the barrel 20. For example, the petals 34 may be sized so that the petals are more readily flexed or bent transversely outward from their closed configuration to allow easy expulsion of the tampon 14 from the barrel 20 and to eliminate the possibility that the petals may in any way provide discomfort to the user. For exemplary purposes only, for example, the petals 34 can have a length of from about 10 mm to about 20 mm, such as from about 14 mm to about 18 mm. The petals 34 generally have a length equal to or greater than an inner diameter of the barrel 20 within the central region 32. For example, a ratio of the petal length to the inner diameter of the barrel 20 can be in the range of from about 1 to about 2, such as from about 1 to about 1.5, such as from about 1 to about 1.25.

In one aspect, the petals 34 are made from a material, have a thickness, and have a size that causes the petals to be bent inward to their closed configuration closest to the outer end or tips of the petals, as opposed to being bent nearer to the bases of the petals. This provides a softer, more flexible feel to the petals 34. In addition, in one embodiment, the petals 34 can have a lower stiffness than the central region 32 of the barrel 20. For example, in one aspect, the petals 34 can have a thickness that is less than the thickness of the central region 32 of the barrel 20. The reduced thickness of the petals 34 allows the petals to be more flexible and pliable to provide a soft, flexible feel and appearance.

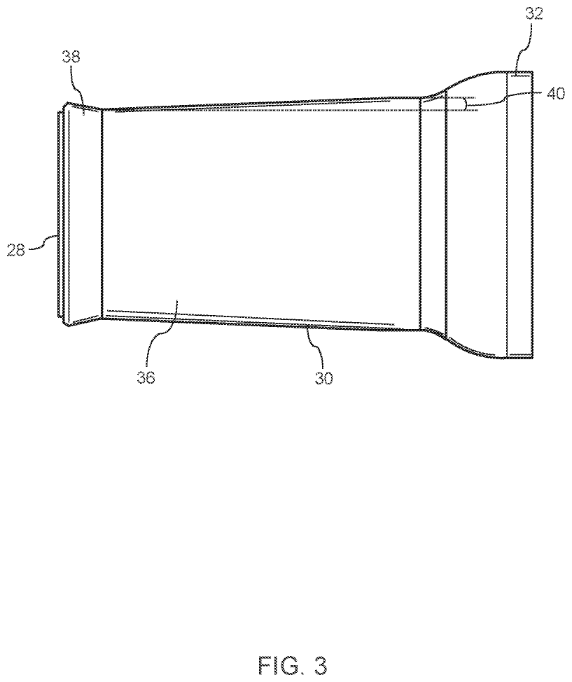

Referring now to FIGS. 3-5, the grip region 30 made in accordance with the present disclosure is shown in greater detail. The grip region 30 of the tampon applicator 12 includes a unique design that has been found to provide excellent grip characteristics while also displaying improved manufacturability, especially when producing the tampon applicator 12 in a single shot injection molding process. In the past, the grip region 30 was typically overmolded with an elastomeric coating. Through the design of the present disclosure, however, an easy to hold and manipulate grip region is produced without the need for overmolding with a different material, such as an elastomeric coating.

As shown in FIG. 3, the grip region 30 includes a tapered section 36 that is adjacent to and terminates with a grip ring 38. The combination of the tapered section 36 and the grip ring 38 have been found well suited for accommodating the fingers and hands of a user.

As shown particularly in FIG. 3, the tapered section 36 extends along the longitudinal axis of the barrel 20 and gradually decreases in diameter from the central region 32 to the grip ring 38. In one aspect, the tapered section 36 includes a continuous, gradual and constant taper.

More particularly, the tapered section 36 tapers at an angle 40 to the longitudinal axis of the barrel 20. The angle 40 is generally less than about 5°. More particularly, the angle when optimized is generally less than about 4°, such as less than about 3°, such as less than about 2.5°. The angle 40 of the taper is generally greater than about 0.5°, such as greater than about 1°, such as greater than about 1.5°. In one particular embodiment, the angle 40 is from about 1.75° to about 2.25°. It was discovered that the above angle optimizes not only the tactile or gripping properties of the grip region 30 but also facilitates removal of the part during the molding process, especially when formed from a polymer material.

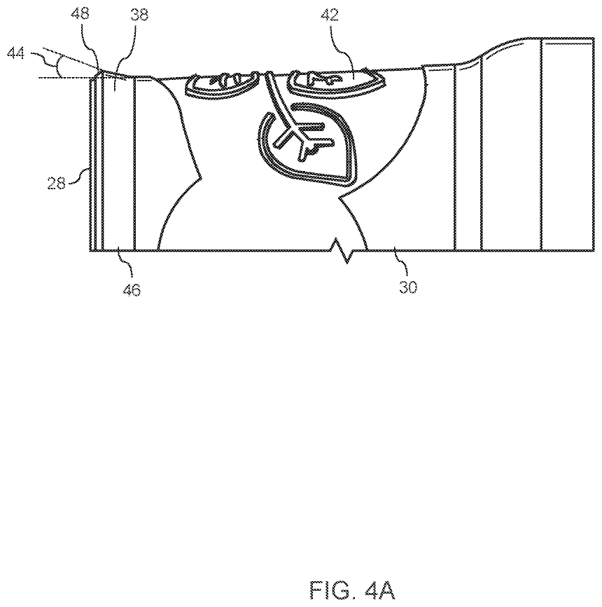

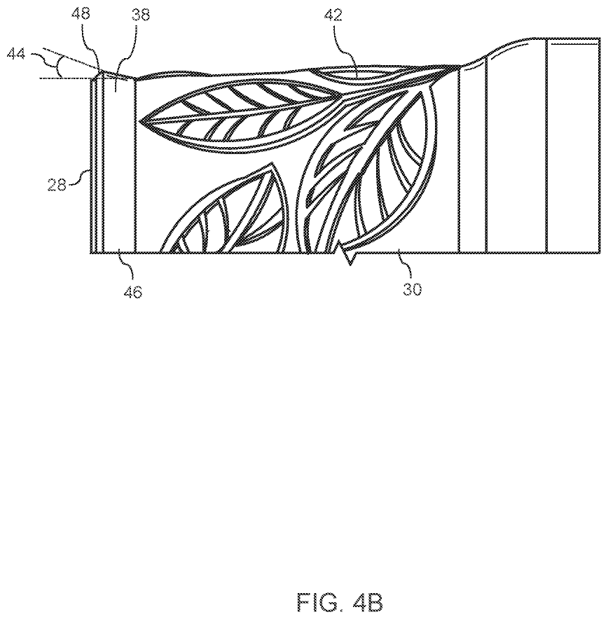

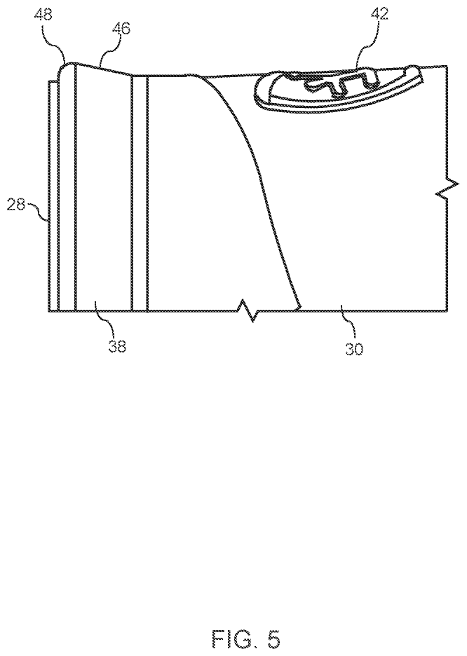

In the embodiment illustrated in FIG. 3, the surface of the grip region 30 is shown as smooth and uniform. If desired, the mold used to produce the barrel 20 can be constructed so as to increase the coefficient of friction of the surface by adding texture. For example, the grip region can include a raised or recessed molded pattern. In one aspect, as shown in FIGS. 4A, 4B, and 5, the grip region 30 can include a raised pattern 42. The raised pattern 42 is for facilitating gripping of the tampon applicator 12. In the embodiment illustrated in FIGS. 4A, 4B, and 5, the raised pattern 42 can also be decorative illustrating, for instance, a leaf design or any other suitable design. The raised pattern 42 includes a pattern of raised sections that makes the gripping region 30 easier to hold and handle.

The length of the tapered section 36 of the grip region 30 can vary depending upon the particular application. For exemplary purposes, in one aspect, the length of the tapered section 36 can be generally less than about 26 mm, such as less than about 24 mm, such as less than about 22 mm, such as less than about 20 mm, such as less than about 18 mm. The length is generally greater than about 12 mm, such as greater than about 14 mm. The tapered section 36 of the grip region 30 can represent generally greater than about 15% of the length of the entire barrel 20, such as greater than about 18%, such as greater than about 20%, such as greater than about 22% of the entire length of the barrel 20, and generally less than about 32%, such as less than about 30%, such as less than about 28% of the entire length of the barrel 20. For example, the length of the barrel 20 can be generally greater than about 65 mm, such as greater than about 68 mm, such as greater than about 70 mm, such as greater than about 72 mm, and generally less than about 95 mm, such as less than about 90 mm, such as less than about 85 mm, such as less than about 80 mm, such as less than about 77 mm. The outside diameter of the tapered section 36 of the grip region 30 is generally from about 8 mm to about 16 mm, such as from about 10 mm to about 14 mm, and can vary from one end of the tapered section 36 to the other end by from about 0.3 mm to about 0.7 mm.

Referring to FIGS. 4A, 4B, and 5, the grip ring 38 is shown in more detail. As shown, the grip ring 38 includes an angled portion 46 adjacent a rounded portion 48. The rounded portion 48 terminates at the outer end 28 of the barrel 20. As shown in FIGS. 4A and 4B, the angled portion 46 of the grip ring 38 can have an angle 44 relative to the longitudinal axis of the barrel 20. The angle 44 is constructed so as to be amenable to molding while also providing a stop for a person's hand or fingers when manipulating the tampon applicator. For example, the angle 44 can be generally greater than about 7°, such as greater than about 9°, such as greater than about 10°, such as greater than about 11° with respect to the longitudinal axis. The angle 44 is generally less than about 17°, such as less than about 16°, such as less than about 15°, such as less than about 14°, such as less than about 13° with respect to the longitudinal axis.

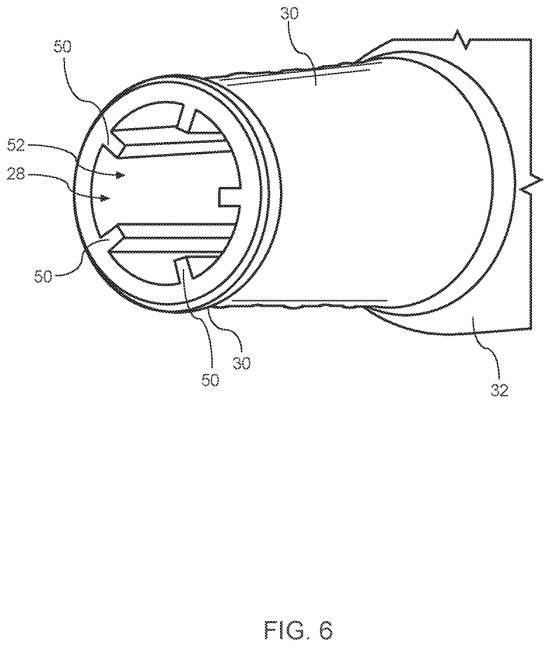

As shown in FIG. 2, the grip region 30 can have an inner diameter and an outer diameter that are generally less than the inner diameter and outer diameter of the central region 32. In addition, as shown in FIGS. 6, 7, and 8, the barrel 20 can also include a plurality of internal ribs 50 that can extend over the grip region 30. For example, the internal ribs 50 can extend along a portion of the length of the tapered section 36, can extend from a first end and to a second end of the tapered section 36, or can extend over the tapered section 36 into the central region 32.

The ribs 50 extend into the barrel 20 to provide support and alignment of the plunger 22 as it moves through the barrel 20. Specifically, the ribs 50 collectively define a longitudinal guide channel 52 through which the plunger 22 extends and is supported by a support surface defined by each of the ribs 50. In this manner, the ribs 50 define an effective diameter that intersects the facing surface of each rib.

In the embodiment illustrated in FIG. 6, the effective diameter of the guide channel 52 is circular. In other embodiments, however, the guide channel 52 may have a non-circular circumference. In this embodiment, the effective diameter of the ribs 50 is the diameter of the largest cylinder that could pass through the guide channel 52 without deformation.

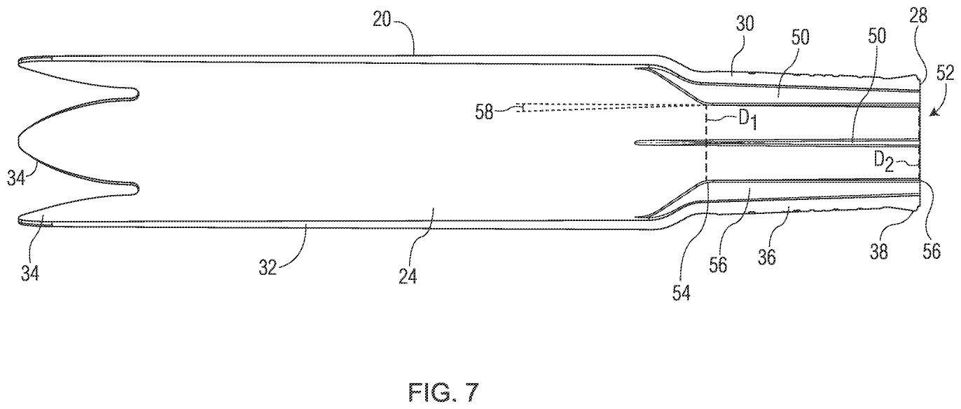

Referring to FIG. 7, the ribs 50 include a leading end 54 and a trailing end 56. The trailing end 56 is adjacent the outer end 28 of the barrel 20. The leading end 54 is oriented towards the insertion end of the tampon applicator 12. In various embodiments, the effective diameter of the guide channel 52 may be the same from the leading end 54 to the trailing end 56. However, in some embodiments, the effective diameter of the guide channel 52 may vary from the leading end 54 to the trailing end 56. For example, as illustrated in FIG. 7, the internal ribs 50 may define a first effective diameter D1 proximate the leading end 54 of the ribs 50 and a second effective diameter D2 proximate the trailing end 56 of the ribs 50. In one embodiment, the first effective diameter D1 can be substantially the same as the second effective diameter D2. In an alternative embodiment, however, the first effective diameter D1 may be greater or less than the second effective diameter D2.

In the embodiment illustrated in FIG. 7, the first effective diameter D1 is greater than and tapers toward the second effective diameter D2. The difference in the effective diameters can be relatively minor but can significantly improve the manner in which the plunger moves through the barrel 20. The design illustrated in FIG. 7 can also facilitate the expulsion of the tampon pledget from the barrel 20 for more consistent application of the tampon pledget to a vaginal canal. The design can also facilitate removal of the part from the mold. The effective diameter can generally be from about 4.5 mm to about 12 mm, such as from about 5.5 mm to about 7 mm, such as from about 6 mm to about 6.8 mm over the length of grip region 30. The difference between the first effective diameter D1 and the second effective diameter D2 can be less than about 0.5 mm, such as less than about 0.4 mm, and generally greater than about 0.1 mm, such as greater than about 0.2 mm.

The ribs 50, as shown in FIGS. 6 and 7, can have any suitable height, width, length or cross-sectional shape. The height of the ribs 50 can be constant or can vary along the longitudinal axis of the barrel 20. As shown in FIG. 7, in one embodiment, the ribs 50 gradually taper from the leading end 54 to the trailing end 56. For example, the taper can form an angle 58 with the longitudinal axis of greater than about 0.1°, such as greater than about 0.3°, such as greater than about 0.4°, and generally less than about 2.5°, such as less than about 2°, such as less than about 1.5°, such as less than about 1°, such as less than about 0.7°. The rib height, in one embodiment, can be generally less than about 3 mm, such as less than about 2 mm, and generally greater than about 0.5 mm, such as greater than about 1 mm, such as greater than about 1.5 mm, depending upon the particular embodiment. As shown in FIG. 7, in the embodiment illustrated, the taper of the ribs 50 is in an opposite direction than the taper of the tapered section 36 of the grip region 30.

In addition to rib height, the rib width can also vary along the longitudinal axis of the barrel 20. For example, as shown in FIG. 7, in one embodiment, the width of the ribs 50 taper from the trailing end 56 to the leading end 54. The taper can be at an angle of generally greater than about 0.3°, such as greater than about 0.5°, such as greater than about 0.7°, and generally less than about 3°, such as less than about 2°, such as less than about 2.5°, such as less than about 2.3°. Tapering the width of the ribs 50 can facilitate insertion of the plunger during assembly and can facilitate mold removal.

The number of ribs 50 contained within the barrel 20 can vary depending upon the particular application. In the embodiment illustrated, the tampon applicator 12 includes more than 4 ribs, particularly 5 ribs. In other embodiments, the tampon applicator 12 can include 6 ribs or 7 ribs. As shown in FIGS. 6 and 7, the ribs can be essentially linear and can be parallel with the longitudinal axis of the barrel 20.

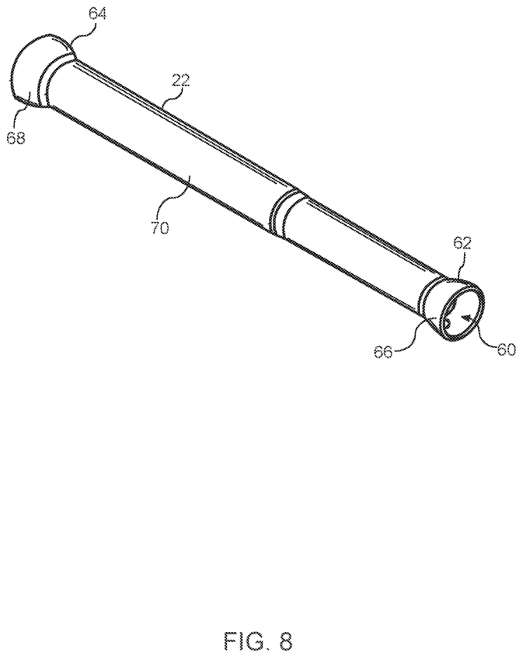

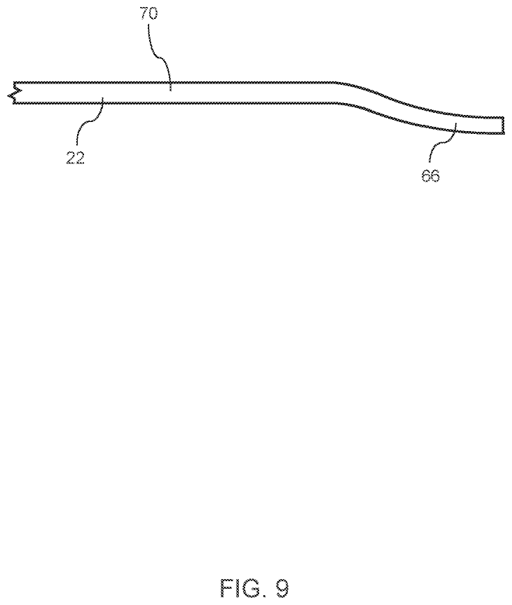

Referring now to FIGS. 8 and 9, the plunger 22 of the tampon applicator 12 is shown in greater detail. The plunger 22 is elongated and may include a hollow channel 60 so that the withdrawal string 18 attached to the tampon 14 can extend out through the plunger 22. The plunger 22 includes a finger-contacting end 62 opposite a tampon-contacting end 64. As shown in FIGS. 1 and 2, the plunger 22 has a length sufficient to extend through the barrel 20 for ejecting a tampon 14 through the inner end 26 of the barrel 20. In this regard, the plunger 22 can have a length that is within about 20% of the length of the barrel 20. For exemplary purposes only, in one embodiment, the plunger 22 can have a length of greater than about 50 mm, such as greater than about 60 mm, such as greater than about 70 mm, and generally less than about 110 mm, such as less than about 100 mm, such as less than about 90 mm, such as less than about 80 mm.

Referring to FIG. 8, the plunger 22 can include an increased outer diameter adjacent the finger-contacting end 62 and an increased outer diameter adjacent the tampon-contacting end 64. For example, the tampon 22 can include a finger flare 66 on one end and a tampon flare 68 on the opposite end. The finger flare 66 and the tampon flare 68 can have any suitable shape such as a flange, ring or bell-shaped configuration or any other suitable shape to facilitate gripping and/or pushing the plunger with a finger or pushing a tampon through the barrel 20. The tampon flare 68 can also serve as a stop to inhibit the tampon-contacting end 64 of the plunger 22 against exiting the barrel 20.

The finger flare 66, for example, may have any suitable effective diameter and the tampon flare 68 may also have any suitable effective diameter. The effective diameter of the finger flare 66 and the tampon flare 68 is equivalent to the outer diameter of the flares having a circular shape. For flares having a non-circular shape, the effective diameter refers to the diameter of the smallest cylinder through which the non-circular flare can pass without deformation.

The finger flare 66 can have an effective diameter greater than the effective diameter of the guide channel 52 defined by the ribs 50. For example, the finger flare 66 may have an effective diameter of at least about 5 mm, such as at least about 5.5 mm, such as at least about 6 mm, such as at least about 6.5 mm, and generally less than about 9 mm, such as less than about 8 mm. In one aspect, the finger flare 66 may have an effective diameter that is at least about 5%, such as at least about 10%, such as at least about 15%, such as at least about 20% larger than the effective diameter of the guide channel 52 at the outer end 28.

Similarly, the tampon flare 68 may have an effective diameter also greater than the effective diameter of the guide channel 52 defined by the ribs 50. For example, the tampon flare 68 can have an effective diameter of at least about 6 mm, such as at least about 7 mm, such as at least about 8 mm, such as at least about 9 mm, and generally less than about 14 mm, such as less than about 12 mm, such as less than about 10 mm. The tampon flare 68, for instance, can have an effective diameter that is at least about 5%, such as at least about 10%, such as at least about 15%, such as at least about 20% larger than the effective diameter of the guide channel 52.

As shown in FIG. 8, the plunger 22 further includes an elongated shaft 70. The elongated shaft 70 can have a constant diameter or can have a tapered diameter as shown in FIG. 8. In the embodiment illustrated in FIG. 8, for instance, the shaft tapers from the tampon-contacting end 64 to the finger-contacting end 62. The shaft 70 also has an effective diameter. The largest effective diameter of the shaft 70 is generally less than the minimum effective diameter of the guide channel 52. In this manner, the shaft can be inserted through the guide channel 52 and be capable of back and forth motion supported by the ribs 50. For example, the largest effective diameter of the shaft 70 can be within about 20%, such as within about 15%, such as within about 10%, such as within about 5%, such as within about 2% of the minimum effective diameter of the internal guide channel 52.

In one embodiment, the tampon flare 68 and/or the finger flare 66 are constructed so as to be relatively flexible. In this manner, during assembly of the tampon product 10, one end of the plunger can be inserted through the internal guide channel 52 without damage to the finger flare 66 or the tampon flare 68. Once one end of the plunger 22 is inserted through the guide channel 52, the plunger assumes a locked position within the tampon applicator 12.

In this regard, in one embodiment, the thickness of the finger flare 66 can diminish and be less than the thickness of the shaft 70. For example, for exemplary purposes only, FIG. 9 illustrates a reduced wall thickness of the finger flare 66. In one embodiment, the thickness of the end of the finger flare 66 can be less than about 0.05 mm, such as less than about 0.08 mm, such as less than about 0.1 mm, such as less than about 0.12 mm the wall thickness of the shaft 70. The difference in wall thickness between the end of the finger flare 66 and the thickness of the shaft 70 is generally less than about 0.25 mm, such as less than about 0.2 mm, such as less than about 0.18 mm, such as less than about 0.15 mm. The wall thickness of the tampon flare 68 can be similar to the wall thickness of the finger flare 66. In this regard, the wall thickness of the tampon flare 68 can also be smaller than the wall thickness of the shaft 70 and can be smaller than the wall thickness of the shaft 70 according to the same dimensions described above with respect to the finger flare 66.

As described above, one of the benefits and advantages of the tampon applicator design of the present disclosure is the ability to produce the tampon applicator in a single one-shot molding step from a single material or polymer composition. By making the tampon applicator from a single polymer composition or material, the tampon applicator can be constructed so as to be completely sustainable, recyclable, biodegradable, compostable, and/or flushable.

In general, the tampon applicator 12 can be made from any suitable material, including paperboard, a polymer or a mixture of polymers. In one aspect, the tampon applicator 12 can be made from a single layer of elastomer. Alternatively, the tampon applicator can be made from recycled polymers or from a polymer that can be recycled. For instance, the tampon applicator can be made from a polyolefin such as, without limitation, polypropylene, polyethylene, low density polyethylene, medium density polyethylene, high density polyethylene, linear low density polyethylene, near low density polyethylene, polyethylene terephthalate, polybutylene terephthalate, a polyamide, or mixtures thereof.

In an alternative embodiment, the tampon applicator can be made from a bio-based polymer, such as a biodegradable and/or flushable polymer. For example, the tampon applicator can be made from polylactic acid, a polyhydroxyalkanoate, or mixtures thereof.

For example, one particularly suitable biodegradable polyester is polylactic acid, which may generally be derived from monomer units of any isomer of lactic acid, such as levorotory-lactic acid (“L-lactic acid”), dextrorotatory-lactic acid (“D-lactic acid”), meso-lactic acid, or mixtures thereof. Monomer units may also be formed from anhydrides of any isomer of lactic acid, including L-lactide, D-lactide, meso-lactide, or mixtures thereof. Cyclic dimers of such lactic acids and/or lactides may also be employed. Any known polymerization method, such as polycondensation or ring-opening polymerization, may be used to polymerize lactic acid. A small amount of a chain-extending agent (e.g., a diisocyanate compound, an epoxy compound or an acid anhydride) may also be employed. The polylactic acid may be a homopolymer or a copolymer, such as one that contains monomer units derived from L-lactic acid and monomer units derived from D-lactic acid. Although not required, the content of one of the monomer units derived from L-lactic acid and the monomer unit derived from D-lactic acid is preferably about 85 mol % or more, in some embodiments about 90 mol % or more, and in some embodiments, about 95 mol % or more. Multiple polylactic acids, each having a different ratio between the monomer unit derived from L-lactic acid and the monomer unit derived from D-lactic acid, may be blended at an arbitrary percentage. Of course, polylactic acid may also be blended with other types of polymers (e.g., polyolefins, polyesters, etc.).

In one particular embodiment, the polylactic acid has the following general structure:

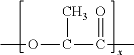

The polylactic acid can have a number average molecular weight (“Mn”) ranging from about 40,000 to about 180,000 grams per mol, in some embodiments from about 50,000 to about 160,000 grams per mol, and in some embodiments, from about 80,000 to about 120,000 grams per mol. Likewise, the polymer also typically has a weight average molecular weight (“Mw”) ranging from about 80,000 to about 250,000 grams per mol, in some embodiments from about 100,000 to about 200,000 grams per mol, and in some embodiments, from about 110,000 to about 160,000 grams per mol. The ratio of the weight average molecular weight to the number average molecular weight (“Mw/Mn”), i.e., the “polydispersity index”, is also relatively low. For example, the polydispersity index typically ranges from about 1.0 to about 3.0, in some embodiments from about 1.1 to about 2.0, and in some embodiments, from about 1.2 to about 1.8. The weight and number average molecular weights may be determined by methods known to those skilled in the art.

Other biodegradable polyesters that may be used to construct the tampon applicator include polycaprolactone, polyesteramides, polyglycolic acid, poly-3-hydroxybutyrate (PHB), poly-3-hydroxyvalerate (PHV), poly-3-hydroxybutyrate-co-4-hydroybutyrate, poly-3-hydroxybutyrate-co-3-hydroxyvalerate copolymers (PHBV), poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-3-hydroxybutyrate-co-3-hydroxyoctadecanoate, and succinate-based aliphatic polymers (e.g., polybutylene succinate, polybutylene succinate adipate, polyethylene succinate, etc.); aliphatic-aromatic copolyesters (e.g., polybutylene adipate terephthalate, polyethylene adipate terephthalate, polyethylene adipate isophthalate, polybutylene adipate isophthalate, etc.), and mixtures thereof.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A tampon applicator comprising:
an elongated barrel having an interior chamber for housing a tampon therein, an outer end and an inner end spaced longitudinally from the outer end, and a grip region generally adjacent the outer end of the barrel;
a plunger extending into the barrel at the outer end thereof and moveable relative to the barrel to expel a tampon from the barrel at the inner end of the barrel; and
wherein the grip region has a first end and a second and opposite end that coincides with the inner end of the barrel, the second end defining a grip ring and wherein the grip region gradually tapers from the first end to the grip ring.

2. A tampon applicator as defined in claim 1, wherein the elongated barrel defines a longitudinal axis and wherein the grip region gradually tapers at an angle with respect to the longitudinal axis of from about 0.5° to about 5°, such as from about 1° to about 2°, such as from about 1.5° to about 2.5°.

3. A tampon applicator as defined in claim 1, wherein the elongated barrel including the grip region is made from a single piece of material.

4. A tampon applicator as defined in claim 1, wherein the elongated barrel including the grip region is made entirely from a single layer of the same polymer composition.

5. A tampon applicator as defined in claim 1, wherein the elongated barrel defines a longitudinal axis and wherein the grip ring flares outwardly from the grip region at an angle to the longitudinal axis of from about 7° to about 17°, such as from about 10° to about 14°, such as from about 11° to about 13°.

6. A tampon applicator as defined in claim 1, wherein the grip region further defines a raised pattern molded into an outer surface of the grip region for facilitating gripping of the tampon applicator.

7. A tampon applicator as defined in claim 1, further comprising a plurality of internal ribs located in the grip region, the plurality of ribs defining a guide channel having an effective diameter and wherein the plurality of internal ribs have a leading end and a trailing end that extend along a longitudinal axis of the elongated barrel.

8. A tampon applicator as defined in claim 7, wherein the internal ribs have a height and wherein the height of the internal ribs is greater at the leading end of the internal ribs than at the trailing end of the internal ribs.

9. A tampon applicator as defined in claim 8, wherein the internal ribs gradually taper from the leading end to the trailing end.

10. A tampon applicator as defined in claim Z, wherein the applicator includes greater than 4 ribs, such as from 5 ribs to 6 ribs.

11. A tampon applicator as defined in claim 1, wherein the plunger has a finger-contacting end, a tampon-contacting end, and an elongated portion therebetween, the plunger further including a finger flare on the finger-contacting end and a tampon flare on the tampon-contacting end, the finger flare, the tampon flare, and the elongated portion all having a thickness and wherein the thickness of the finger flare is less than the thickness of the elongated portion.

12. A tampon applicator as defined in claim 11, further comprising a plurality of internal ribs located in the grip region, the plurality of ribs defining a guide channel having an effective diameter and wherein the plurality of internal ribs have a leading end and a trailing end that extend along a longitudinal axis of the elongated barrel, and wherein the tampon flare has an effective flare diameter that is larger than the effective diameter of the guide channel.

13. A tampon applicator as defined in claim 1, wherein the tampon applicator is entirely formed from a bio-based polymer.

14. A tampon applicator as defined in claim 13, wherein the bio-based polymer comprises a polylactic acid, a polyhydroxyalkanoate, or mixtures thereof.

15. A tampon applicator as defined in claim 1, wherein the elongated barrel includes at least two petals each having a base and extending longitudinally of the barrel to a tip of the petal wherein the petal tips define the inner end of the barrel, the petals being configured relative to each other and to the barrel from a closed configuration in which the petals generally close the inner end of the barrel and an open position in which the petals are moved generally transversely outward to form an exit opening at the inner end of the barrel upon movement of the plunger to expel a tampon from the barrel at the inner end.

16. A tampon applicator as defined in claim 15, wherein the barrel includes 5 or 6 petals.

17. A tampon applicator as defined in claim 1, wherein the tampon applicator is made from a single layer of an elastomeric material.

18. A tampon product comprising a tampon applicator as defined in claim 1 and further comprising a tampon pledget contained within the elongated barrel.

19. A tampon product as defined in claim 18, further comprising a drawstring extending from the tampon pledget and through the plunger.

20. A tampon product as defined in claim 18, wherein the tampon pledget comprises cotton fibers.
